(19) United States
(12) Patent Application Publication
(10) Pub. No.: US 2007/0231390 A1
(43) Pub. Date: Oct. 4, 2007

(54) Formulations including hygroscopic compounds

(75) Inventors: Aaron Dely, Lighthouse Point, FL (US); Unchalee Lodin, North Miami Beach, FL (US); Avinash Nangia, Lincoln, RI (US)

Correspondence Address: DAVIDSON, DAVIDSON & KAPPEL, LLC, 485 SEVENTH AVENUE, 14TH FLOOR, NEW YORK, NY, 10018, US

(73) Assignee: Andrx Labs, LLC, Davie, FL (US)

(21) Appl. No.: 11/391,739

(22) Filed: Mar. 29, 2006

(51) Int. Cl. A61K 9/24

(52) U.S. Cl. 424/473; 514/52; 514/277; 514/423; 514/557; 424/725; 514/165; 514/35; 514/276

(57) ABSTRACT

The invention is directed to dosage forms suitable for the administration of hygroscopic active agents.

### FIELD OF THE INVENTION

The invention is directed oral dosage forms including a hygroscopic compound and processes for the preparation of the dosage forms. In particular, the invention is directed to oral dosage forms including a salt of valproic acid and processes for the preparation the dosage forms.

### BACKGROUND OF THE INVENTION

Hygroscopic compounds pose problems when included in oral solid dosage forms, in that they absorb moisture from the atmosphere and/or the solid dosage form itself, possibly leading to an unstable, brittle and/or deformed dosage form, which may be susceptible to breakage and leakage.

Salt forms of valproic acid are examples of hygroscopic compounds which pose problems for formulators in manufacturing stable oral solid dosage forms.

Valproic acid and the salts of valproic acid are useful for treating various forms of epilepsy as well as certain other disorders (e.g., petit mal, monoclonic seizures, generalized and partial motor seizures, absence and infantile spasms, partial epilepsy, bipolar disorders (psychotic disorders) and migraine). Although valproic acid or its salts have known utility as anti-convulsants, a number of problems are associated in formulating them in a solid form.

In particular, the sodium salt of valproic acid, sodium valproate, is useful for treating epileptic phenomena. This drug is effective for its intended therapy, however, sodium valproate is hygroscopic and liquifies rapidly. These properties can interfere with the manufacture and release of the drug from a dosage form, and are drawbacks in the management of epilepsies and other disorders which may be treated with sodium valproate.

Various attempts have been made to formulate moisture-stable solid valproic acid and valproic acid salt formulations. U.S. Pat. No. 5,049,586 describes conventional (immediate-release) formulations of valproic acid containing fillers, disintegrants, binders and lubricants. The lubricated granulate described therein is said to be a dry, non-hygroscopic mixture which is said to be suitable for use in forming compressed tablets or for filling capsules. The formulation is asserted to be moisture stable and to need no protective coating. According to the '586 patent, the formulations are prepared using a wet granulation step.

U.S. Pat. No. 6,204,255 describes formulations of sodium valproate in combination with cyclodextrins.

U.S. Pat. No. 5,688,510 describe in certain embodiments en bloc processing of certain hygroscopic active agents with excipients in an extruder.

U.S. Pat. No. 5,212,326 and WO 96/23491 describe a stable, non-hygroscopic, solid complex prepared from valproic acid and its salts, which is a single crystalline entity consisting of one molecule each of valproic acid and sodium valproate salt. The methods described in U.S. Pat. No. 5,212,326 and WO 96/23491 require reaction between valproic acid and its salt to produce a new entity.

A need exists for a solid oral dosage form containing a hygroscopic active agent, and in particular, that is easily produced and is stable. There is a further need for such formulations to be sustained and/or delayed release formulations.

### OBJECTS AND SUMMARY OF THE INVENTION

It is an object of the present invention to provide an oral solid dosage form that is suitable for the oral administration of a hygroscopic active agent.

It is a further object of certain embodiments of the present invention to provide an oral solid dosage form for the controlled delivery of a hygroscopic active agent over an extended period of time.

It is a further object of certain embodiments of the present invention to provide a stable oral solid dosage form comprising a hygroscopic active agent.

It is a further object of certain embodiments of the present invention to provide a controlled release dosage form comprising a hygroscopic active agent that provides therapeutic levels of the active agent to an animal or human in need of such treatment over a twelve hour to twenty-four hour period.

It is a further object of certain embodiments of the present invention to provide a method for treating epilepsy by administering to an individual in need of such treatment a therapeutically effective amount of a pharmaceutically acceptable salt of valproic acid in a dosage form suitable for oral administration.

Other objects and features of this invention will be more apparent to those of ordinary skill in the art from the following specification, taken in conjunction with the drawings and the accompanying claims.

In accordance with the above-mentioned objects and others, the present invention is directed in part to an oral solid dosage form including a directly compressed core composition including a hygroscopic active agent and a non-polymeric pharmaceutically acceptable excipient wherein the oral solid dosage form is suitable for oral administration to a human patient.

In certain embodiments, the invention is directed to an oral solid dosage form including a dry granulated compressed core composition including a therapeutically effective amount of a hygroscopic active agent and a non-polymeric pharmaceutically acceptable excipient wherein the oral solid dosage form is suitable for oral administration to a human patient.

In certain embodiments, the invention is further directed to an oral solid dosage form including a directly compressed bi-layer core composition including a first layer comprising a first portion of a hygroscopic active agent and first pharmaceutically acceptable excipient and a second layer comprising a second portion of the hygroscopic active agent and a second pharmaceutically acceptable excipient wherein the hygroscopic active agent is included in the dosage form in a therapeutically effective amount. The first and second pharmaceutically acceptable may be the same or different pharmaceutically acceptable excipient(s).

In certain embodiments, the invention is further directed to an oral solid dosage form including a dry granulated and compressed bi-layer core composition including a first layer comprising a first portion of a hygroscopic active agent and first pharmaceutically acceptable excipient and a second layer comprising a second portion of the hygroscopic active agent and a second pharmaceutically acceptable excipient wherein the hygroscopic active agent is included in the dosage form in a therapeutically effective amount. The first and second pharmaceutically acceptable may be the same or different pharmaceutically acceptable excipient(s).

In certain embodiments, the invention is further directed to an oral solid dosage form including a directly compressed tri-layer core composition including a first layer comprising a first portion of a hygroscopic active agent and first pharmaceutically acceptable excipient, a second layer comprising a second portion of the hygroscopic active agent and a second pharmaceutically acceptable excipient, and a third layer comprising a third portion of the hygroscopic active agent and a third pharmaceutically acceptable excipient, wherein the hygroscopic active agent is included in the dosage form in a therapeutically effective amount. The first, second, and third pharmaceutically acceptable excipients may be the same or different pharmaceutically acceptable excipient(s).

In certain embodiments, the invention is further directed to an oral solid dosage form including a dry granulated and compressed tri-layer core composition including a first layer comprising a first portion of a hygroscopic active agent and first pharmaceutically acceptable excipient, a second layer comprising a second portion of the hygroscopic active agent and a second pharmaceutically acceptable excipient, and a third layer comprising a third portion of the hygroscopic active agent and a third pharmaceutically acceptable excipient, wherein the hygroscopic active agent is included in the dosage form in a therapeutically effective amount. The first, second, and third pharmaceutically acceptable excipients may be the same or different pharmaceutically acceptable excipient(s).

In certain embodiments, the invention is further directed to a controlled release oral solid dosage form including a directly compressed core composition comprising a therapeutically effective amount of a hygroscopic active agent and a pharmaceutically acceptable excipient, a semi-permeable membrane surrounding the directly compressed core, and at least one passageway disposed in the semi-permeable membrane, wherein the dosage form provides a controlled release of the hygroscopic active agent over a period of about 12 to about 24 hours upon exposure of the dosage form to an environmental fluid.

In certain embodiments, the invention is further directed to a controlled release oral solid dosage form including a dry granulated compressed core composition comprising a therapeutically effective amount of a hygroscopic active agent and a pharmaceutically acceptable excipient, a semi-permeable membrane surrounding the directly compressed core, and at least one passageway disposed in the semi-permeable membrane, wherein the dosage form provides the controlled release of said hygroscopic agent over a period of about 12 to about 24 hours upon exposure to an environmental fluid.

In certain embodiments, the invention is further directed to an oral tablet formulation including a therapeutically effective amount of a hygroscopic active agent and a non-polymeric pharmaceutically acceptable excipient; the tablet being produced by forming a dry blend of the hygroscopic active agent and the non-polymeric pharmaceutically acceptable excipient and directly compressing the dry blend to form the oral tablet formulation. In certain embodiments, the blend is dry granulated and the compressed to form the oral tablet formulation.

In certain embodiments, the invention is further directed to a sustained release oral tablet formulation including a therapeutically effective amount of a hygroscopic active agent and a pharmaceutically acceptable excipient; the tablet being produced by forming a dry blend of the hygroscopic active agent and the non-polymeric pharmaceutically acceptable excipient, directly compressing the dry blend to form a tablet core formulation, and coating the tablet core formulation with a semi-permeable membrane to form the sustained release oral tablet formulation. Preferably, at least one passageway is disposed in the semi-permeable membrane.

In certain embodiments, the present invention is further directed to a process for preparing an oral solid dosage form including as an active ingredient, a hygroscopic active agent, comprising mixing the hygroscopic active agent and a pharmaceutically acceptable excipient to preferably form a homogeneous or substantially homogenous mixture; directly compressing the mixture to provide a core composition for use in an oral solid dosage form, wherein the amount of said excipient is sufficient relative to the amount of said hygroscopic active agent to produce a stable oral solid dosage form. Alternatively, the mixture may be dry granulated and then compressed into the core formulation.

In certain embodiments, the present invention is further directed to a method of treating a medical condition in a human patient, the method comprising the step of orally administering an oral solid dosage form as described herein to a human patient in need of treatment with the hygroscopic active agent included in the oral solid dosage form.

In certain preferred embodiments, the core composition of the present invention is a matrix formulation having the active agent dispersed therein.

In certain further preferred embodiments, the core composition of the present invention is a bilayer core composition having the active agent dispersed in both layers of the bilayer composition.

In certain further preferred embodiments, the core composition of the present invention is a trilayer core composition having the active agent dispersed in all three layers of the trilayer composition.

In certain embodiments, the oral dosage form of the present invention is preferably in the form of a tablet.

In certain embodiments, the dosage form of the invention is suitable for administration on a once-a-day basis. When administered on a once-a-day basis, the daily dose may vary depending on the clinical needs of the patient. Such daily dose may be contained in one controlled-release dosage form of the invention, or may be contained in more than one such dosage form. For example, when the active agent is sodium valproate, a controlled-release dosage form may be formulated to contain sodium valproate in an amount equivalent to 100, 250 or 500 mg valproic acid; and a combination of these dosage forms may be administered together to provide a desired once-a-day valproic acid dose.

In certain preferred embodiments of the present invention, the dosage form comprises a pharmaceutically acceptable salt of valproic acid as the hygroscopic active agent. Such salt forms include, for example and without limitation, sodium valproate, potassium valproate, calcium valproate, and mixtures thereof. In most preferred embodiments, the salt form is sodium valproate.

In certain embodiments, wherein the hygroscopic active agent is sodium valproate, the present invention is further directed to a method for treating epilepsy and/or affective illness by administering a therapeutically effective amount of sodium valproate, in a dosage form described herein to an individual in need of such treatment.

In certain embodiments wherein the hygroscopic active agent is sodium valproate, the present invention is further directed to a method for treating and/or preventing migraine by administering a therapeutically or prophylactically effective amount of sodium valproate, in a dosage form described herein to an individual in need of such treatment.

In certain embodiments, wherein the oral dosage form includes sodium valproate in a controlled release oral dosage form, the core comprising sodium valproate and pharmaceutically acceptable excipients is surrounded by a semi-permeable membrane. The semi-permeable membrane provides for the controlled release release of sodium valproate from the core for a period of more than 8 hours, preferably for a period of from about 12 to about 24 hours, preferably providing twice-a-day or once-a-day therapy of sodium valproate.

The term “semi-permeable membrane”, is defined for purposes of the present invention as a membrane that is permeable to the passage of an fluid, such as aqueous and biological fluid, in the environment of use, including the gastrointestinal tract; and impermeable to the passage of the hygroscopic active agent.

The term “sustained release” and “controlled release” are used interchangeably in this application and are defined for purposes of the present invention as the release of the drug from the dosage form at such a rate that when a dose of the drug is administered in the sustained release or controlled-release form, blood (e.g., plasma) concentrations (levels) of the drug are maintained within the therapeutic range but below toxic levels over a period of time from about 12 to about 24 hours.

A “hygroscopic” active agent is defined for purposes of the present invention as an active agent that readily absorbs water (usually from the atmosphere).

The terms “effective amount” or “therapeutically effective amount” of an active agent is defined for purposes of the present invention as an amount of the agent at least sufficient to provide the desired therapeutic effect.

The term “environmental fluid” is defined for purposes of the present invention as e.g., an aqueous solution, such as that used for in-vitro testing or gastrointestinal fluid.

### DETAILED DESCRIPTION

The present invention is directed to an oral solid dosage form including a directly compressed core comprising a hygroscopic compound. Alternatively, the core may be a dry granulated compressed core comprising a hygroscopic compound. The compressed core may be further formulate into a controlled release oral dosage form, or alternatively may be employed as an immediate release oral dosage form.

It will be appreciated by those of ordinary skill in the art that a directly compressible core has advantages as it represents an efficient tabletting process, namely just mixing the ingredients and then compressing them, thus alleviating the need for the intermediate granulation and drying steps necessary in other tabletting procedures.

Direct compression is regarded as a relatively quick process in which the powdered materials are directly compressed without changing the physical and chemical properties of the drug. The active ingredient and pharmaceutically acceptable excipients are blended in e.g., a twin-shell or similar blending apparatus before being compressed into cores or tablets. This type of mixing is generally preferred in preparing pharmaceutically acceptable dosage forms. Thereafter the mixture is compressed into core compositions.

Dry granulation has the advantage of alleviating the need for the use of a granulating solvent and the drying steps necessary with wet granulation.

In certain preferred embodiments, the oral dosage form of the present invention comprises a compressed core in the form of a bi-layered or tri-layered core composition.

For bi-layered core formulations, the core formulations preferably have a first layer and a second layer. In certain embodiments the first layer is referred to as the top layer and the second layer is referred to as the bottom layer. Preferably, the bottom layer of the formulation is the layer which is compressed in the bottom or lower punch of the compression tooling, and the top layer is the layer which is compressed in the top or upper punch of the compression tooling. The bilayered formulations of the present invention include the active ingredient in both layers of the formulation. However, the top layer of the bilayer formulation includes a greater percentage by weight of active ingredient per layer than the bottom layer of the bilayer formulation. Preferably, the inclusion of a greater percentage by weight of active in the top layer, and a lesser percentage by weight of the active in the bottom layer, contributes to an improved compressing of the core composition (e.g., due to decreased sticking to the lower punch).

With reference to FIG. 1, in certain embodiments, a core composition 10 of the present invention is in the form of a bi-layer composition, having has a top layer 11 and a bottom layer 12.

For tri-layered core formulations, the formulations preferably have a first layer, a second layer and a third layer. In certain embodiments the first layer is referred to as the top layer, the second layer is referred to as the bottom layer and the third layer is referred to as the middle layer. Preferably, the bottom layer of the formulation is the layer which is compressed in the bottom or lower punch of the compression tooling, the top layer is the layer which is compressed in the top or upper punch of the compression tooling, and the middle layer is the layer that is compressed in between the top and bottom layers. Preferably, the middle layer has a greater percentage by weight of the active agent per layer than the top and bottom layers of the core. In certain embodiments of the tri-layered core formulations, the top and bottom layers may contain the same percentage by weight of the active agent or different percentage by weight of the active agent.

With reference to FIG. 2, in certain embodiments, a core composition 20 of the present invention, is in the form of a tri-layer composition having a top layer 21, a bottom layer 22, and a middle layer 23.

The compressed core compositions can be administered as immediate release oral dosage forms (e.g., as a tablet) or can be formulated to be controlled release oral dosage forms. In certain embodiments, wherein the oral dosage forms of the invention are controlled release oral dosage forms the compressed core composition is coated with a semi-permeable membrane.

Preferably, the core of the present invention includes various pharmaceutically acceptable excipients which allow for the preparation of a directly compressible core formulation or a dry granulated compressible core formulation. Preferably the excipients for use in the present invention are non-polymeric pharmaceutically acceptable excipients.

Examples of pharmaceutically acceptable excipients that may be used in the core formulations of the present invention are described in the Handbook of Pharmaceutical Excipients, American Pharmaceutical Association (1986), incorporated by reference herein.

Typical pharmaceutically acceptable excipients for inclusion in the core composition are inert diluents or fillers, lubricants, binders, glidants, and combinations thereof.

Preferably, where an inert diluent is included in the core composition of the present invention, the inert diluent is included in an amount of from about 25% to about 75% by weight of the core composition and more preferably from about 30% to about 40% of the weight of the core composition.

Suitable diluents or fillers include, but are not limited to, lactose (e.g., monohydrate, spray-dried monohydrate, anhydrous and the like), sucrose, dextrose, mannitol, sorbitol, starch, dihydrated or anhydrous dibasic calcium phosphate, calcium carbonate, calcium sulfate, and others as known in the art. More preferably, free-flowing diluents which can improve blend flow are: spray-dried lactose monohydrate, agglomerated free-flowing lactose monohydrate, granulated lactose monohydrate, roller dried lactose monohydrate, direct compression lactose, anhydrous, direct compression sucrose, co-crystallized sucrose and modified dextrins, spray-dried dextrates, coarse dextrose, agglomerated dextrose, spray-dried maltodextrin, fine granular maltodextrin, spray-dried maltose, spray dried mannitol, granular mannitol, spray- dried sorbitol, coarse sorbitol, direct compression fructose co- dried with starch, pregelatinized corn starch, free-flowing grades of dibasic calcium phosphate, dihydrate and free-flowing grades of dibasic calcium phosphate, anhydrous.

A most preferred diluent is lactose. Preferably, a pharmaceutically acceptable lactose in anhydrous form which demonstrates an increase in compressibility, has good binding characteristics and provides sufficient hardness to tablets formed in accordance with the invention is provided as the preferred filler in the formulations of the invention. Preferably, the use of lactose as a filler enables the formulation used in the method of the invention to form hard, strong tablets at low machine pressure which reduces instances of chipping and capping of the tablets as well as reducing the friability of the tablets.

Preferably, the core composition includes at least one glidant, at least one lubricant or combination thereof. The glidants and lubricants may be any suitable glidants and lubricants preferably contribute to the compressibility, flowability and homogeneity of the formulation and preferably minimize segregation

Preferably, where a lubricant is included in the core composition of the present invention, the lubricant can be included in an amount of from about 0.5 to about 3% by weight, or from about 1% to about 2% by weight, of the core composition.

Examples of lubricants are magnesium stearate, glyceryl monostearate, stearic acid, glycerylbehaptate, calcium stearate, oleic acid, caprylic acid, magnesium isovalerate, calcium laurate, magnesium palmitate, behenic acid, glyceryl behenate, glyceryl stearate, zinc stearate, sodium lauryl sulfate, magnesium lauryl sulfate, sodium oleate, sodium stearyl fumarate, DL-leucine, colloidal silica, and others as known in the art. A preferred lubricant is magnesium stearate.

Preferably the inclusion of a lubricant in the core prevents adhesion of the core material to the surface of the dies and punches, reduces interparticle friction, facilitates the ejection of the cores from the die cavity and/or improves the rate of flow of the mixture of active and pharmaceutically acceptable excipient.

Preferably where a binder is included in the core composition of the present invention, the binder can be present in an amount from about 0.5% to about 10%, preferably from about 1% to about 3% by weight of the core composition.

Suitable binder materials include, but are not limited to, gelatin, sugars (including sucrose, glucose, dextrose and maltodextrin), waxes, natural and synthetic gums, mixtures thereof, and the like.

Preferably where a glidant is included in the core composition of the present invention, the glidant can be present in an amount of from 0.1% to about 10%, preferably from about 2% to about 8% by weight, more preferably from about 1% to about 3%, by weight of the core composition.

Preferably the glidant improves the flow characteristics of the mixture of the active agent and pharmaceutically acceptable excipient.

Examples of glidants include, but are not limited to colloidal silicon dioxide, talc, corn starch, mixtures thereof, and the like. Colloidal silicon dioxide is preferred.

If desired, other ingredients, such stabilizers and antiadherants, which are conventionally used for pharmaceutical formulations, may be included in the present formulations. Other optional ingredients include coloring and flavoring agents which are well known in the art.

In certain preferred embodiments, wherein the core composition is a bi-layer composition, the top layer preferably includes from about 45 to about 75 percent by weight of the hygroscopic active agent, from about 15 to about 40 percent by weight of the inert diluent (e.g., lactose, mannitol, maltodextrin, compressed sugar), from about 1 to about 8 percent by weight of glidant (e.g., talc, silicon dioxide), and from about 0.5 to about 3 percent by weight of lubricant (e.g., magnesium stearate); and the bottom layer preferably includes from about 10 to about 20 percent by weight of the hygroscopic active agent, from about 75 to about 90 percent by weight of the inert diluent (e.g., lactose, mannitol, maltodextrin, compressed sugar), from about 1 to about 8 percent by weight glidant (e.g., talc, silicon dioxide), and from about 0.5 to 3 percent by weight of lubricant (e.g., magnesium stearate).

In certain preferred embodiments, wherein the core composition is a tri-layer composition, the top layer preferably includes from about 10 to about 60 percent by weight of the hygroscopic active agent, from about 20 to about 55 percent by weight of the inert diluent (e.g., lactose, mannitol, maltodextrin, compressed sugar), from about 0 to about 10 percent by weight of glidant (e.g., talc, silicon dioxide), and from about 0.25 to about 5 percent by weight of lubricant (e.g., magnesium stearate); and the bottom layer preferably includes from about 10 to about 60 percent by weight of the hygroscopic active agent, from about 20 to about 55 percent by weight of the inert diluent (e.g., lactose, mannitol, maltodextrin, compressed sugar), from about 0 to about 10 percent by weight glidant (e.g., talc, silicon dioxide), and from about 0.25 to 5 percent by weight of lubricant (e.g., magnesium stearate), and the middle layer preferably includes preferably includes from about 10 to about 60 percent by weight of the hygroscopic active agent, from about 20 to about 55 percent by weight of the inert diluent (e.g., lactose, mannitol, maltodextrin, compressed sugar), from about 0 to about 10 percent by weight glidant (e.g., talc, silicon dioxide), and from about 0.25 to 5 percent by weight of lubricant (e.g., magnesium stearate).

In certain preferred embodiments, the multi-layer tablet formulation allows for the different distribution of the active agent in the different layers and reduces the sticking of the tablet to the compression tool punches, most preferably the lower punch. Reduced sticking is desirable as sticking to the punches may lead to an increase in the ejection force of a tableting device and cause the tablet device to stop.

The core composition of the present invention provides a suitable compositioin for the administration of a hygroscopic active agent, which is included in the core composition in a therapeutically effective amount.

Hygroscopic active agents for use in accordance with the present invention include any pharmaceutical therapeutically active ingredient having a tendency to pick up water from the environment by any mechanism such as absorption, adsorption and the like. Examples of hygroscopic active agents include for example and without limitation, salt forms of valproic acid (e.g., sodium valproate), metformin HCl, omeprazole, diclofenac sodium, diethylcarbamazine citrate, betahistine mesylate, carpronium chloride, tolazoline HCl, reserpilic acid dimethylaminoethyl ester dihydrochloride, carbachol, choline theophyllinate, hexamethonium bromide, panthenol, dexamethazone phosphate disodium salts, oxtriphylline, procainamide hydrochloride, gemfibrozil, disopyramide phosphate, fenoprofen calcium, atenolol, piracetam, carbamazepine, tetracycline hydrochloride, oxytetracycline hydrochloride, rifamprin, lincomycin HCl, clindamycin HCl, cefaclor, cefadroxil, cephrabine, thiamine hydrochloride, ascorbic acid, acetyl salicylic acid, methocarbamol, methyldopa, sulindac, desipramine HCl, ranitidine HCl, ethionamide, meprobamate, captopril, aminophylline, pramiracetam, 3-phenoxypyridine monosulphate, cyanocobalamin, L-proline, calcium chloride, plant extracts (mallot cortex extract), and combinations thereof.

Preferably the hygroscopic active agent is included in the core composition in an amount of from about 25% to about 60%, preferably from about 35% to about 55% by weight of the core composition.

In a preferred embodiment, the hygroscopic active agent for use in accordance with the present invention is a pharmaceutically acceptable salt form of valproic acid, most preferably sodium valproate.

Preferably, the solid dosage form contains sodium valproate in an amount equivalent to from about 50 to about 1200 mg of valproic acid, and more preferably, from about 100 to about 650 mg of valproic acid. In preferred embodiments, the amount of sodium valproate used is equivalent to about 500 mg valproic acid.

In certain embodiments, the core composition is a tablet composition suitable for oral administration. In certain embodiments, the tablet composition may optionally be coated with a moisture barrier film, to further improve the non-hygroscopic properties of the composition as discussed below.

Alternatively, the core composition is further processed to provide for a controlled release of the active agent over an extended period of time. In certain embodiments, prior to coating the core with a membrane, the core may be coated with a pharmaceutically acceptable film-coating, e.g., for stability purposes (e.g., coated with a moisture barrier) or for process purposes to further improve the non-hygroscopic properties of the directly compressed core composition. For example, the core may be overcoated with a film coating, preferably containing a pigment and a barrier agent, such as hydroxypropylmethylcellulose and/or a polymethylmethacrylate. An example of a suitable material which may be used for such a hydrophilic coating is hydroxypropylmethylcellulose (e.g., Opadry®), or a combination of hydrophilic coatings may also be used. Optionally the core may be overcoated with a solution of polyvinyl alcohol or other materials of the like. Any pharmaceutically acceptable manner known to those skilled in the art may be used to apply the coatings. For example, the coating may be applied using a coating pan or a fluidized bed. In certain embodiments, the pharmaceutically acceptable film-coating around the core, e.g., for stability purposes, does not affect or does not substantially effect the release of the active agent from the core.

In certain embodiments, wherein a controlled release of the active agent over an extended period of time is desired, a semi-permeable membrane is coated over the compressed core composition. The semi-permeable membrane is preferably a polymeric membrane that provides the controlled release of the active agent. Materials that are useful in forming the membrane are alkylcelluloses (e.g., ethylcellulose), acrylic or methacrylic polymers or copolymers, cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate, cellulose acetate butyrate, or combinations thereof. Other suitable polymers are described in U.S. Pat. Nos. 3,845,770, 3,916,899, 4,008,719, 4,036,228 and 4,111,201 which are incorporated herein by reference.

Preferably, the cellulose ester in the semi-permeable membrane is cellulose acetate. Various grades of cellulose acetate, which differ in their acetyl content and degree of polymerization, are commercially available, and may be used in the present invention. In certain embodiments, the semi-permeable membrane includes a methacrylic acid copolymer which is preferably an ammoniomethacrylate copolymer, such as poly(ethyl acrylate, methyl methacrylate, trimethylammonioethyl methacrylate chloride)

In certain embodiments, the membrane includes or be further coated with an enteric material. As enteric coating material polymers one or more, separately or in combination, of the following can be used; e.g. solutions or dispersions of methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose, shellac, zein or other suitable enteric coating layer polymer(s). Some preferred commercial enteric coating materials are EUDRAGIT® 100-55, EUDRAGIT® L 30 D-55, EUDRAGIT® L 100, and EUDRAGIT® S 100.

In certain embodiments, any combination of the aforementioned polymers can be used in the semi-permeable membrane.

In certain embodiments, the membrane further includes a flux enhancing agent. Preferably, the flux enhancing agent increases the volume of fluid imbibed into the core to enable the dosage form to dispense substantially all of the active agent through the passageway and/or the porous membrane. The flux enhancing agent can be a water soluble material or an enteric material. Some examples of the preferred materials that are useful as flux enhancers are sodium chloride, potassium chloride, sucrose, sorbitol, mannitol, polyethylene glycol (PEG), propylene glycol, hydroxypropyl cellulose, hydroxypropyl methycellulose, hydroxypropyl methycellulose phthalate, cellulose acetate phthalate, polyvinyl alcohols, methacrylic acid copolymers, and mixtures thereof. A preferred flux enhancer is polyethylene glycol.

In certain embodiments, the flux enhancing agent comprises approximately 0 to about 40% of the total weight of the coating, most preferably about 2% to about 20% of the total weight of the coating. The flux enhancing agent dissolves or leaches from the membrane to form paths in the membrane for the fluid to enter the core and dissolve the active ingredient.

In certain embodiments, the semi-permeable membrane may further contain a plasticizer such as glycerol, propylene glycol, polyethylene glycol, sorbitol, diethyl phthalate, mineral oil, triacetin, triethyl citrate, and the like. The plasticisers are generally used in an amount ranging from about 0.01% to about 1% by weight of the tablet. Some commonly known plasticizers include adipate, azelate, enzoate, citrate, stearate, isoebucate, sebacate, triethyl citrate, tri-n-butyl citrate, acetyl tri-n-butyl citrate, citric acid esters, polaxamer (e.g., Lutrol®, commercially available from BASF), and those described in the Encyclopedia of Polymer Science and Technology, Vol. 10 (1969), published by John Wiley & Sons. The preferred plasticizers are polaxamer, triacetin, acetylated monoglyceride, grape seed oil, olive oil, sesame oil, acetyltributylcitrate, acetyltriethylcitrate, glycerin sorbitol, diethyloxalate, diethylmalate, diethylfumarate, dibutylsuccinate, diethylmalonate, dioctylphthalate, dibutylsebacate, triethylcitrate, tributylcitrate, glyceroltributyrate, and the like. Depending on the particular plasticizer, amounts of from 0 to about 25%, and preferably about 2% to about 15% of the plasticizer can be used based upon the total weight of the coating. In certain preferred embodiments, the plasticizer is poloxamer.

In certain embodiments, the semi-permeable membrane further includes a pharmaceutically acceptable excipient as described herein, or other pharmaceutically acceptable excipients known in the art, including polymeric pharmaceutically acceptable excipients.

In certain embodiments, the membrane is further coated with a pharmaceutically acceptable film-coating, e.g., for stability purposes (e.g., coated with a moisture barrier) or for process purposes as described above. For example, the membrane may be overcoated with a film coating, preferably containing a pigment and a barrier agent, such as hydroxypropylmethylcellulose and/or a polymethylmethacrylate. An example of a suitable material which may be used for such a hydrophilic coating is hydroxypropylmethylcellulose (e.g., Opadry®, commercially available from Colorcon, West Point, Pa.). In addition, other suitable materials are Povidone K30, PEG 3350, or the like. Any pharmaceutically acceptable manner known to those skilled in the art may be used to apply the coatings. For example, the coating may be applied using a coating pan or a fluidized bed.

In certain preferred embodiments of the present invention, the oral dosage form contains at least one passageway in the semi-permeable membrane. As used herein the term passageway includes an aperture, orifice, bore, hole, weaken area or an erodible element such as a gelatin plug that erodes to form an osmotic passageway for the release of the valproic acid, pharmaceutically acceptable salt thereof, amide thereof, or derivative thereof from the dosage form. A detailed description of the passageway can be found in U.S. Pat. Nos. such as 3,845,770, 3,916,899, 4,034,758, 4,063,064, 4,077,407, 4,088,864, 4,783,337 and 5,071,607 (the disclosures of which are hereby incorporated by reference).

In certain embodiments the passageway can be formed by drilling, including mechanical and laser drilling, through the membrane. Passageways and equipment for forming passageways are disclosed in U.S. Pat. Nos. 3,845,770, 3,916,899, 4,063,064, and 4,088,864. In certain embodiments, a passageway is drilled in each side of the tablet, forming a tablet having two passageways.

In other embodiments, the passageway is formed by making an indentation onto the core prior to the membrane coating to form a weakened area of the membrane at the point of the indentation. Prior to coating the core with the membrane, the core may be indented. The indentation can be made during compression by an indentation pin located on the press punch. Alternatively, the core can be indented after compression. By “indented” it is meant that there is a depression in the core of the present invention.

In certain embodiments, the semi-permeable membrane coating around the core is less than 10% of the total weight of the dosage form, preferably the membrane coating around the core will be from about 1% to about 7%, preferably from about 2% to about 6%, most preferably from about 3% to about 5% based on the total weight of the formulation.

Preferably, wherein the oral dosage form is a controlled release oral dosage form, the release of the drug from the dosage form is controlled by the membrane surrounding the core and the passageway disposed therein.

The compositions of the present invention may be prepared as solid dosage forms such as tablets, caplets, and capsules. In a preferred embodiment the oral dosage form is a controlled release tablet.

Preferably, the pharmaceutical composition remains non-hygroscopic under relative humidity of from about 30% to about 75% at 40° C. in closed conditions and more preferably from about 30% to about 60% at 25° C. in closed conditions.

In certain further embodiments, the present invention is further directed to a method of preparing the oral dosage forms described herein.

The method comprises combining the hygroscopic active agent, and at least one excipient described herein to form a directly compressible formulation. The formulation is directly compressed (or dry granulated and then compressed) to form the hygroscopic active agent core compositions, which can be administered as an oral tablet formulation, can be placed in a capsule and administered as a capsule formulation, or can be coated with a semi-permeable membrane and optionally other coatings as described herein to provide for the controlled release of the active agent over an extended period of time.

The method of the invention does not require wet granulation and the core composition is to be directly compressed (or dry granulated and then compressed) using any suitable compression apparatus useful for forming pharmaceutical grade tablets. Preferably, the cores are compressed using any suitable rotary tablet press such as a Stokes, Manesty, and the like. The cores formed preferably are compressed to achieve a hardness of from about 5 to about 30 kp, from about 15 to about 30 kp, or from about 10 to about 20 kp.

Prior to compression, the formulation, as described above, may be combined by blending, mixing, stirring, shaking, tumbling, rolling or by any other method of combining the powdered components (e.g., the active agent and pharmaceutically acceptable excipients) preferably to achieve a homogeneous blend. However, it is most preferred that the excipients and the hygroscopic active ingredient are combined using a V-blender, twin shell blender or similar apparatus capable of functioning under preferred low shear conditions. Impellers, knives, or other rotating blades may be used to facilitate combining the formulation ingredients.

In certain embodiments, to prepare the dry blend, the various components may be weighed, delumped and combined except for the lubricating agent (e.g., magnesium stearate). The mixing may be carried out for a sufficient period of time to produce a homogeneous blend, and then the lubricant may be added. Afterwards, the final mixing may be carried out. The dry blend may be stored for later use or compressed on suitable equipment.

In certain embodiments, wherein the blend is dry granulated, preferably the blend is dry granulated using a compacting mill such a Chilsonator (Fitzpatrick), Roller Compactor (Vector), Compactor Mill (Allis-Chalmers), or the like. Preferably the compacting mill increases the particle size and reduces the sticking to the compression tool.

The compressed cores are suitable for administration as a conventional immediate release oral dosage form, or are coated to provide for the controlled release of the active agent over an extend period of time.

There are several types of coating equipment used to coat the core compositions. One type is the pan coater in which tablets are rotated in a pan and coating solution is applied to the tablets (e.g., core compositions) as tablets tumble in the pan. Another coating process involves suspending the tablets (e.g., core compositions) in a column of air while the coating solution is sprayed onto the tablets (fluid bed process). One example of this is the Wurster column coating process. The tablet or core composition may be coated by any known process and the manner of application is not limited to any particular equipment.

The present invention further provides a method of treating a medical condition in a human patient, the method including the step of orally administering a non-hygroscopic highly stable pharmaceutical composition for release of a hygroscopic active agent into the bloodstream at a therapeutically effective level, wherein the composition includes a pharmaceutically effective amount of a hygroscopic active agent and a pharmaceutically acceptable excipient. In certain embodiments, the method of treating a medical condition in a human patient includes providing therapeutically effective levels of the hygroscopic active agent over an extended period of time (e.g., from about 12 to about 24 hours).

In certain preferred embodiments, the present invention is further directed to a method of treating epilepsy, a psychotic disorder or a migraine headache comprising orally administering an oral solid dosage form comprising a pharmaceutically acceptable salt form of valproic acid and a pharmaceutically acceptable excipient to a patient in need of such therapy. In certain embodiments, the method of treating epilepsy, a psychotic disorder, or a migraine headache in a human patient includes providing therapeutically effective levels of the pharmaceutically acceptable salt form of valproic acid over an extended period of time (e.g., from about 12 to about 24 hours). Although the preferred salt is sodium valproate, any other hygroscopic salt or derivative of valproic acid that is suitable for oral administration, or mixtures of valproic acid salts and derivatives can be used.

Typically, to treat seizure disorders, sodium valproate is formulated in a tablet or other dosage form in amounts of 10-40 mg/kg body weight per day, preferably 15-30 mg/kg body weight per day. For adults, the daily dose is typically 20 mg/kg body weight per day. For children and infants, the daily dose is typically 25 mg/kg body weight per day.

When a sustained release dosage form is to be administered, the daily dosage of sodium valproate or other solid derivative of valproate acid is formulated in a sustained release composition to be released slowly to maintain therapeutic levels of valproic acid in patients blood between about 50 to about 100 μg/ml.

The daily dose can be formulated in a single tablet, or more than one tablet, depending on the daily dose of the valproate salt, the final weight of the composition and the number of times the formulation is to be administered.

In certain embodiments of the present invention, controlled release compositions can be formulated such that, in vitro, preferably from about 5% to about 40% of the active ingredient is released after 2 hours, preferably from about 10% to about 50% is released after 6 hours, approximately from about 30% to about 90% is released after 8 hours and approximately from about 50% to about 100% is released within 12 to 24 hours (test parameters USP type I apparatus).

Immediate release compositions can be formulated such that in vitro approximately 70% of the drug is released in one hour.

### DESCRIPTION OF CERTAIN PREFERRED EMBODIMENHTS

The following example illustrates various aspects of the present invention. It is not to be construed to limit the claims in any manner whatsoever.

### EXAMPLE 1

In Example 1, a sodium valproate core formulation was prepared and is listed in table I below:

TABLE 1

| Material | % w/w | mg/tablet |
|---|---|---|
| Sodium Valproate, EP | 64.00 | 576.0 |
| Anhydrous Lactose, NF, | 30.00 | 270.0 |
| direct tableting | | |
| Colloidal Silicon Dioxide, | 2.00 | 18.00 |
| NF (Cab-O-Sil M5) | | |
| Talc USP (AlTalc 500v) | 3.00 | 27.00 |
| Magnesium Stearate | 1.00 | 9.00 |
| Totals | 100.0 | 900.0 |

The core (or tablet) formulation of Example 1 was prepared as follows:
1. The Sodium valproate, EP was milled using a #313 (20 mesh) screen;
2. The Sodium valproate, EP and Cab-O-Sil were blended in a V-blender for 10 minutes.
3. The Sodium valproate, EP and Cab-O-Sil was passed through a Comil equipped with a 30 mesh screen.
4. The Sodium valproate, EP, Cab-O-Sil, and Talc were passed through a 20 mesh screen;
5. The Sodium valproate, EP, Cab-O-Sil, and Talc were blended in a V-blender for 10 minutes;
6. The Lactose was screened through a 20 mesh, and blended with the blend for 15 minutes;
7. The magnesium stearate was then added to the blend and blended for 5 minutes.
8. The blend was compressed using a Fette press to provide for the cores (or tablets). To facilitate this step, desiccant packs were placed onto the tablet press turret in an attempt to control the localized moisture from adhering to the surface of the punch which would lead to “sticking”; humidity was controlled (in the room <30%) to minimize “sticking”; punches were heated (in an oven) prior to use to facilitate evaporation of atmospheric moisture from the punches; and warm air was blown onto the punches during the process to facilitate atmospheric moisture evaporation.

The core (tablet) formulation of Example 1 had excellent hardness (greater than 25 kp and “sticking” to the tablet press was minimal on the upper punch and the lower punch.

### EXAMPLE 2

In Example 2, a sodium valproate core formulation was prepared and is listed in table 2 below:

TABLE 2

| Material | % w/w | mg/tablet |
|---|---|---|
| Sodium Valproate, EP | 54.00 | 576.0 |
| Anhydrous Lactose, NF, | 40.00 | 426.7 |
| direct tableting | | |
| Colloidal Silicon Dioxide, | 2.00 | 21.33 |
| NF (Cab-O-Sil M5) | | |
| Talc USP (AlTalc 500v) | 3.00 | 32.00 |
| Magnesium Stearate | 1.00 | 10.67 |
| Totals | 100.0 | 1066.7 |

The core (or tablet) formulation of Example 2 was prepared as described in Example 1 above.

The core of Example 2 was prepared at a 1025 mg weight as a 1066 mg tablet was not achievable with the cams available on the Fette press. Tablet hardness was greater than 30 kp (30 to 40 kp) ant the “sticking” was minimal on the upper and lower punches.

### EXAMPLE 3

In Example 3, a controlled release sodium valproate oral dosage form was prepared as is listed in table 3 below

| Material | % w/w | mg/tablet |
|---|---|---|
| Sodium Valproate, EP | 50.94 | 576.0 |
| Anhydrous Lactose, NF, | 31.75 | 359.0 |
| direct tableting | | |
| Colloidal Silicon Dioxide, | 1.769 | 20.00 |
| NF (Cab-O-Sil M5) | | |
| Talc USP (AlTalc 500v) | 2.653 | 30.00 |
| Magnesium Stearate | 1.327 | 15.00 |
| Hypromellose 2910, USP | 1.547 | 17.50 |
| (Methocel E5 Premium) | | |
| Hydroxypropyl Cellulose | 1.032 | 11.67 |
| (Klucel EF) | | |
| Magnesium Stearate, NF | 1.105 | 12.50 |
| Ethyl Alcohol SDA 3A 190 | * | * |
| Proof (Ethanol) | | |
| Cellulose Acetate, NF 398- | 2.771 | 31.33 |
| 10 | | |
| Hydromellose Phthalate NF | 1.108 | 12.53 |
| 24% (HP-50) | | |
| Poloxamer 188 NF (Lutrol | 1.000 | 10.96 |
| F-68) | | |
| Acetone, NF | * | * |
| Opadry Color | 3.000 | 33.92 |
| Candeilla Wax Powder, | 0.030 | 0.339 |
| FCC | | |
| Totals | 100.03 | 1130.8 |

The above ingredients were blended in accordance with the procedures of Example 1.

Many other variations of the present invention will be apparent to those skilled in the art and are meant to be within the scope of the claims appended hereto.

1-52. (canceled)

53. A controlled release oral solid dosage form comprising:
(a) a directly compressed core composition comprising a therapeutically effective amount of a hygroscopic active agent and a pharmaceutically acceptable excipient;
(b) a semi-permeable membrane surrounding the directly compressed core; and
(c) at least one passageway disposed in the semi-permeable membrane; said dosage form providing a controlled release of the hygroscopic active agent over a period of about 12 to about 24 hours upon exposure of the dosage form to an environmental fluid.

54. The solid dosage form of claim 53, wherein said core is a dry granulated compressed core.

55. The controlled release oral solid dosage form of claim 53, wherein the core is a bi-layer core composition having the active agent dispersed in both layers of the bi-layer composition.

56. The controlled release oral solid dosage form of claim 53, wherein the core is a tri-layer core composition having the active agent dispersed in three layers of the tri-layer core composition.

57. The controlled release oral solid dosage form of claim 53, which is suitable for administration on a once-a-day basis.

58. The controlled release oral solid dosage form of claim 53, wherein the semi-permeable membrane comprises an enteric polymer.

59. The controlled release oral solid dosage form of claim 53, wherein the semi-permeable membrane comprises a polymer selected from the group consisting of an alkylcellulose, acrylic or methacrylic polymer or copolymer, cellulose ester, cellulose diester, cellulose triester, cellulose ether, cellulose ester-ether, cellulose acylate, cellulose diacylate, cellulose triacylate, cellulose acetate, cellulose diacetate, cellulose triacetate, cellulose acetate propionate, cellulose acetate butyrate, and combinations thereof.

60. The controlled release oral solid dosage form of claim 53, wherein the semi-permeable membrane further comprises a flux enhancing agent.

61. The controlled release oral solid dosage form of claim 60 wherein the flux enhancing agent is selected from the group consisting of sodium chloride, potassium chloride, sucrose, sorbitol, mannitol, polyethylene glycol (PEG), propylene glycol, hydroxypropyl cellulose, hydroxypropyl methycellulose, hydroxypropyl methycellulose phthalate, cellulose acetate phthalate, polyvinyl alcohols, methacrylic acid copolymer, polaxamer, and mixtures thereof.

62. A process for preparing an oral solid dosage form comprising:
(a) mixing a hygroscopic active agent and a non-polymeric pharmaceutically acceptable excipient to form a mixture;
(b) directly compressing the mixture to provide an oral solid dosage form suitable for oral administration.

63. The process of claim 62 further comprising coating the oral solid dosage form with a semi-permeable membrane.

64. The process of claim 63 further comprising forming at least one passageway in the semi-permeable membrane.

65. An oral solid dosage form comprising:
a directly compressed core composition of at least one layer consisting essentially of a therapeutically effective amount of a hygroscopic active agent and a non-polymeric pharmaceutically acceptable excipient.

66. The oral solid dosage form of claim 65, wherein said core is prepared by dry granulation.

67. The oral solid dosage form of claim 65, wherein said core comprises more than one layer.

68. The oral solid dosage form of claim 67, wherein each layer is comprised of a therapeutically effective amount of a hygroscopic active agent and a non-polymeric pharmaceutically acceptable excipient.

69. The oral solid dosage form of claim 65, wherein said hygroscopic active agent is selected from the group consisting of a salt form of valproic acid, metformin HCl, omeprazole, diclofenac sodium, diethylcarbamazine citrate, betahistine mesylate, carpronium chloride, tolazoline HCl, reserpilic acid dimethylaminoethyl ester dihydrochloride, carbachol, choline theophyllinate, hexamethonium bromide, panthenol, dexamethazone phosphate disodium salts, oxtriphylline, procainamide hydrochloride, gemfibrozil, disopyramide phosphate, fenoprofen calcium, atenolol, piracetam, carbamazepine, tetracycline hydrochloride, oxytetracycline hydrochloride, rifamprin, lincomycin HCl, clindamycin HCl, cefaclor, cefadroxil, cephrabine, thiamine hydrochloride, ascorbic acid, acetyl salicylic acid, methocarbamol, methyldopa, sulindac, desipramine HCl, ranitidine HCl, ethionamide, meprobamate, captopril, aminophylline, pramiracetam, 3-phenoxypyridine monosulphate, cyanocobalamin, L-proline, calcium chloride, a plant extract, and combinations thereof.

70. The solid dosage form of claim 65, wherein the active agent is a pharmaceutically acceptable salt isomer or derivative of valproic acid.

71. The oral solid dosage form of claim 65, wherein the active agent is sodium valproate

72. The oral solid dosage form of claim 71, wherein the sodium valproate is in an amount equivalent to from about 100 to about 650 mg of valproic acid.
